(19) United States
(12) Patent Application Publication
(10) Pub. No.: US 2016/0215053 A1
(43) Pub. Date: Jul. 28, 2016

(54) CXCL12 (CHEMOKINE (C-X-C MOTIF) LIGAND 12) AND IGFBP2 INHIBITORS FOR THE APPLICATION IN THE TREATMENT OF DIABETES MELLITUS ASSOCIATED PANCREATIC CANCER

(71) Applicants: Gábor Firneisz, Budapest (HU); Ralf Jesenofsky, Bad Dürkheim (DE); Matthias Löhr, Weinheim (DE)

(72) Inventors: Gábor Firneisz, Budapest (HU); Ralf Jesenofsky, Bad Dürkheim (DE); Matthias Löhr, Weinheim (DE)

(21) Appl. No.: 14/915,590

(22) PCT Filed: Aug. 27, 2014
(86) PCT No.: PCT/HU2014/000077

§ 371 Date: Feb. 29, 2016

(30) Foreign Application Priority Data
Aug. 30, 2013 (HU) PI300509

(51) Int. Cl. C07K 16/28; A61K 31/395; A61K 39/00; A61K 31/713

(52) U.S. Cl. CPC C07K 16/2866; A61K 31/713; A61K 31/395; A61K 39/0005; C07K 16/2848; C07K 2317/21; A61K 2039/53; C07K 2317/24

(57) ABSTRACT

The subject of the invention is the application of CXCL12 (Chemokine (C-X-C motif) Ligand 12) and IGFBP2 inhibitors for the treatment of diabetes mellitus associated pancreatic cancer. The core of this invention is the discovery that the chronically increased glucose levels (chronic hyperglycemia) could may an important role in the development of the pancreatic cancer and that the development of the pancreatic cancer due to chronic hyperglycemia or an already developed pancreatic cancer may be prevented/inhibited/delayed by the inhibition of CXCL12 and IGFBP2. In addition the subject of the invention is the production of inhibitors for the application as a treatment of diabetes mellitus associated pancreatic cancer and pharmaceutical drugs containing the inhibitors.

### TECHNICAL FIELD

The subject of the invention is the application of CXCL12 (Chemokine (C-X-C motif) Ligand 12) and IGFBP2 inhibitors for the treatment of diabetes mellitus associated pancreatic cancer.

### BACKGROUND ART

### 1. The Current Standing of the Technics

### 1. a)

### Diabetes Mellitus and Pancreatic Cancer

Diabetes mellitus (DM) is a major public health challenge not only as a risk factor for cardiovascular diseases, but also because it has been linked to a number of cancer types including pancreatic cancer (PaC), one of the deadliest cancer types. Epidemiologic studies established clear evidence between DM and PaC.(1-6)

A meta-analysis of 35 cohort studies in 2011 assessed whether DM is a causative factor or a consequence of PaC.(1) The study confirmed that DM is associated with an increased risk of PaC in both males and females (with the highest risk of PaC found among patients diagnosed within less than 1 year) and strongly supported that DM is not only an early manifestation, but also an etiologic factor of pancreatic cancer.

Six years earlier Huxley and co-workers conducted a meta-analysis based on 17 case-control and 19 cohort or nested case-control studies with information on 9.220 individuals with pancreatic cancer, support a modest causal association between T2DM and PaC.(4)

Perrin and colleagues investigated the incidence of pancreatic cancer in a cohort of more than 37 thousand women for 28-40 years after they give birth in 1964-1976. Information on glucose metabolism in pregnancy was available and the authors concluded that women with a history of gestational diabetes mellitus (GDM) showed 7.1×-fold increase in relative risk of pancreatic cancer with a time-frame of 14-35 years between the onset of gestational diabetes in pregnancy and the later diagnosis of pancreatic cancer (the median age at diagnosis of pancreatic cancer was 58 years for women with previous GDM).(7) Later another Israeli study used a population-based historical cohort design (more than 185 thousand women out of which 11.264 were diagnosed with GDM) and identified a similarly high and statically significant relative risk of GDM on PaC (7.06×-fold) despite a relatively small number of pancreatic cancer cases and shorter time-frame.(8)

A ten-year prospective cohort study of 1,298,385 Asians aged 30 to 95 years was conducted by Jee and coworkers. They analyzed during the 10 years of follow-up more than 20 thousand cancer deaths and concluded that elevated fasting serum glucose levels and a diagnosis of diabetes are independent risk factors for several major cancers, and the risk tends to increase with an increased level of fasting serum glucose. By cancer site, the association was strongest for pancreatic cancer (HR: 1.9-2.05, men-women, respectively) and mortality from pancreatic cancer was associated with a significant increase in risk among women with fasting serum glucose levels above 5 mmol/L.(9) The results of this study suggest that even glucose levels in the upper range of normal could be associated with an increased risk of some cancers, including pancreatic cancer.

Li and coworkers analyzed the data of 397,783 adults in the USA who participated in their Risk Factor Surveillance System and had valid data on diabetes and cancer, they concluded that after adjustment for potential confounders, diabetic men had 4.6×-fold higher adjusted prevalence ratio for pancreatic cancer.(5)

Taken together the link between DM and PaC is likely to be mutual, nevertheless causal association has been established from medical information that may be categorized in 4 types:

In addition to direct analyses of studies with T2DM patients (1, 2, 4) or cancer patients with diabetes mellitus(5), results obtained from two other distinct forms of diabetes also supports causal association.

The results from studies with pregnant women with gestational diabetes mellitus (GDM) that is a disease, where diabetes resolves immediately after delivery in the majority of patients, but years/decades later T2DM will develop in 50-70% of patients with previous GDM pregnancies(7, 8).

As a third line of evidence the results from studies on young adults with type 1 diabetes mellitus (T1DM) that also showed higher risk for PaC that because of the—extreme infrequency of pancreatic cancer in young people—suggested that type 1 diabetes (such as GDM) precedes pancreatic cancer not the other way around. (6)

Fourth type of information is obtained from the study where fasting serum glucose levels were directly assessed in the follow-up of more than 1 million human subjects. According to the results the elevated fasting serum glucose levels is an independent risk factor for several cancers—by cancer site—the strongest for pancreatic cancer, and the risk tends to increase with an increased level of fasting serum glucose.(9)

Pancreatic cancer, of which 90% is ductal adenocarcinoma, still poses an unresolved clinical challenge. The overall 5 years survival is only 5-6% (6-23-9-2% depending on the stage at diagnosis: 2002-2008: all stages-local-regional-distant in the US, respectively) and due to the fact that still the majority of patients die within a year.(10) The survival data observed besides the current oncological treatments clearly indicates that there is a high need for newer treatment options in pancreatic cancer.

A number of biological mechanisms have been suggested to explain the potentially causal relationship between DM and pancreatic cancer (immunologic, hormonal and metabolic), but the relationship has not yet been fully uncovered.

### 1. b) Tumor Associated Fibroblasts

### Tumor Microenvironment and Pancreatic Stellate Cells—Pancreatic Cancer

Pancreatic stellate cells (PSCs) were discovered in the 1980s and PSCs could only be isolated and kept in cell culture as a result of the work by Bachem and Apte in 1998.(11, 12) In the healthy pancreas the PSCs are located in close proximity to the basal surface of the acinar cells, their spatial localization reminds to other localization of pervivascular pericytes in other organs (e.g.: breast). In case of healthy circumstances, PSCs are in resting condition that is phenotypically characterized by the presence of retinoid containing vacuoles in the cytoplasm. Pancreatic stellate cells account for 4-7% of the parenchymal cells in the healthy pancreas.(13)

The stromal, desmoplastic reaction, characteristic for majority of pancreatic tumors, serves as evidence for the participation of PSCs in tumor development.

In addition to that the potentially least aggressive mucinous type of pancreatic cancer is associated with the lowest degree of stromal reaction (14), according to Japanese authors' pathological observations the alpha smooth muscle actin (aSMA) positivity that correlates with the degree of desmoplastic reaction clearly correlated with the biological agressivity of pancreatic ductal adenocacrinoma (PDAC): the higher aSMA expression in the pancreatic tissue resected due to PDAC was associated with a lower survival, based on the analysis of more than 100 PDAC patients.(15)

While the activated stellate cells are the major source of the extracellular matrix (ECM) protein production and deposition in certain diseases of the liver and in the pancreas, in other organs fibroblasts are responsible for this. The consequences of the activation of this system, a process driven originally by transforming growth factor beta (TGF-β), that has been evolved among others to enhance wound healing seemingly are catastrophic in cancer disease. Cancer-associated fibroblasts have come under scrutiny in the recent years/decade and the majority of authors agree that the tumor-associated fibroblasts are unique cellular elements of the stromal tumor-microenvironment and have an essential role in cancer development and growth.(16)

The trans-differentiation of PSCs from resting to active state might be induced in addition to TGF-β, a major determinant by other molecular factors such as: PDGF-β, TNF-α, IL1, IL6, IL8, Activin-A, oxidative stress (ROS), acetaldehyde, ethanol (13)—(certain molecules, e.g.: PDGF-β has a more pronounced PSC proliferation promoting effect, than TGF-β (17), meanwhile in case of other molecular stimuli the ECM production promoting effect or the inhibitory effect on PSC apoptosis might be more asserted.

The factors that induce activation/trans-differentiation of PSCs that may be confirmed using ‘activation’ markers (including cell proliferation, αSMA expression, loss of retinoid droplets, or ECM protein production) in part based on the review publication by Apte and co-workers (18) are summarized in Table 1.

TABLE 1
Factors that induce PSC activation/trans-differentiation

| Factor | Effect on PSC | Reference number |
|---|---|---|
| TGFβ1 | increased ECM synthesis | (19, 20) |
| | increased αSMA expression | (20) |
| Activin A | increased αSMA expression | (21) |
| PDGF | increased proliferation | (19, 20) |
| | increased FN synthesis | |
| bFGF | increased proliferation | (20) |
| | and increased FN synthesis | |
| TNFα | increased αSMA expression | (20, 22) |
| | increased proliferation and | |
| | type-1 collagen production | |
| IL-1 | increased αSMA expression | (22) |
| IL-10 | increased type-1 collagen production | (22) |
| TGFα | increased proliferation, migration | (23) |
| | and MMP1 expression | |
| Prosztaglandin | increased proliferation, migration | (24) |
| E2 | and ECM synthesis | |
| CCK, | increased collagen synthesis, | (25) |
| gasztrin | decreased proliferation | |
| Galektin-1 | increased proliferation | (26) |
| | and type-1 collagen production | |
| Ethanol, | increased proliferation and | (27), (28) |
| Acetaldehide | type-1 collagen production | |
| | increased αSMA expression | (27, 29) |
| ROS | increased αSMA expression, | 29 (30, 31) |
| | proliferation and type-1 collagen | |
| | production | |

The activated PSCs are characterized by high mitotic index, contraction ability (myofibroblast-like), and in addition to ECM synthesis the increased expression of different receptors (PDGF-R, TGF-Rs, ICAM-1), MMP and TIMP secretion (ECM-turnover), and the secretion of neurotrophic factors/transmitters: NGF, Ach, different growth factors and cytokines (PDGF-β, FGF, TGFβ1, CTGF, IL-1s, IL-6, IL8, RANTES, MCP-1, ET-1, VEGF, SDF-1).(13)

Pancreatic stellate cells induce the process of EMT characterized by epithelial marker loss (e.g.: loss of E-cadherin) in cancer cells, therefore promote the progression of the pancreatic tumor.(32)

Experimental data suggested not only that antitumor immunity was suppressed by stromal cells expressing fibroblast activation protein (FAP)-alpha, but also that an agent targeting FAP-expressing cells (nonredundant, immune-suppressive component of the tumor microenvironment) could increase the success of eliminating solid tumors and metastatic cells—by awakening the immune response against the tumor.(33)

In the liver and in the pancreas these FAP, alpha SMA expressing cells are not regular fibroblasts, rather activated stellate cells.

When exposed to stimuli the stellate cells—that are in resting state in the healthy pancreas—transform to an activated myofibroblast-like state, that is characteristic both for pancreatic cancer and for chronic pancreatitis.(18)

During activation PSCs are losing their cytoplasmic retinoid droplets, contractile elements (e.g.: smooth muscle actin, SMA) occur in their cytoplasm, and PSCs may respond both with proliferation or with secretion of ECM components.

In addition to the synthesis of ECM proteins (e.g.: type-1 and type-3 collagens) the activated stellate cells release a variety of different growth factors and cytokines which on one hand may perpetuate their activation state and on the other hand have an effect on the biological characteristics determining the malignant features of pancreatic tumor cells (promote their proliferation).(34-37)

In addition to the direct effect of activated PSCs on pancreatic cancer cells, they protect tumor cells from the immune response and promote vascularization, resulting in increased tumor survival, growth and metastatic spread. (34-37)

The effect of pancreatic stellate cells on the proliferation of cancer cells may evolve in two ways: both via direct cell-cell contact or via microenvironmental, paracrine effects. This phenomenon is difficult to study in the human body in vivo, therefore the observations made using human pancreatic stellate cells or immortalized stellate cell lines are substantial.

Fujita and his group concluded that the direct cell contact between the tumor cell and the activated cancer-associated PSC has an important role in the determination of the proliferation of cancer cells and also important in the understanding of the tumor-stroma interactions.(38)

The role of the soluble factors secreted by PSC is also substantial, due to that when pancreatic cancer cell line was treated by the cell culture media of PSCs, in addition that the proliferation of PSCs increased significantly, a dramatic 400% increase was observed in the migration assay and a 300% increase was described in the invasion assay compared to the migratory and invasive capability of cancer cells which were not treated with the PSC cell culture media. These unfavourable effects at cell level (promotion of cancer cell proliferation, invasion, migration)—that in the practice may correspond to the phenomenon behind the tumor and metastasis formation—could be suspended by inhibiting one of the receptors of the Chemokine (C-X-C motif) Ligand 12 (CXCL12, alias SDF-1), using AMD3100, that is in clinical trials in other diseases.(39)

These phenomena may have a role in the observation that when pancreatic cancer cells were inoculated into an in vivo system (orthotopic, athymic pancreatic cancer animal model) not only the desmoplastic reaction became more pronounced, but also the size of the later size of the original tuboth mor (approximately 20-fold increase), and number, incidence of the regional and the distant metastases (increased: liver: from 35% to 85%, mesenterium: from 21% to 57%-ra, diaphragm: from 7% to 35%), furthermore the number of the organs affected by metastasis (e.g.: kidney increased from 0% to 50%) was determined by that the cancer cell inoculation happened together with the inoculation of pancreatic cancer-associated human PSCs or without the stellate cells during the operation.(37)

Due to the study design (“sex mismatch”) that allowed that male human PSCs (in part cancer-associated) and female cancer cells were together operated into female athymic mice these experiments provided evidence (with the identification of chromosome Y in the metastasis), that the pancreatic cancer and stellate cells got there to the metastatic site together and not only the cancer cells alone! Furthermore, when the number of identified (using FISH method) cells with chromosome Y (so the number of inoculated hPSCs) was compared to the number of 100 cytokeratin positive cells (number of inoculated cancer cells) in the metastatic nodule, the authors concluded that the mean ratio of PSCs to metastatic cancer cells in the metastases is: 5.6 to 1.(37)

The observation—that in pancreatic cancer cells (BxPC3 cells) treated with the cell culture media of human PSCs (hPSC-CM) the gemcitabine (Gemzar) induced apoptosis was decreased: proportion of cancer cells undergoing apoptosis changed from 38.9% to 9.4% (approximately ¼) as a result of hPSC-CM treatment—may be highly important from the point of the everyday clinical practice.(40) This phenomenon may—in part—serve as an explanation that why even the gemcitabine based treatment results in ductal pancreatic cancer are miserable and also provide evidence that PSCs may release soluble substances that induces resistance of the pancreatic cancer cells against the drugs which applied according to the current chemotherapeutic protocols (and also against irradiation).(40)

It is to be highlighted that according to the current standing of the technics there was no role for glucose and/or chronic hyperglycemia in the secretion of the above mentioned soluble substances by PSCs, therefore this process—according to the current standing of the technics—has not been related to diabetes mellitus, that is characterized by chronically higher than normal glucose levels.

A number of authors raised the possibility that the progression of pancreatic cancer is fundamentally determined by the minor proportion of tumor cells, which may be considered as cancer stem cells.(41-43) The cancer stem cells in the pancreas account only for 0.2-0.8% of the tumor cells, the tumorogenic potential of this special cancer cell subpopulation possessing characteristic phenotypic markers (CD44+, CD24+, ESA+) is 100-fold compared to the non-tumorogenic cancer cells (this group of cells is also more resistant against treatments) and the injection of only 100 such cells into NOD/SCID mice is sufficient for the development of a tumor that histologically may not be differentiated from the original human tumor.(44) However the mechanisms maintaining the “stem cell character” are yet not fully elucidated. Japanese authors came to the conclusion that pancreatic stellate cells actively participate also in this process: treatment of pancreatic cancer cells with PSC cell culture media enhanced the development of stem cell-like phenotype, the spheroid-forming ability of cancer cells and induced the expression of cancer stem cell-related genes (ABCG2, Nestin, L1N28), suggesting that PSCs may be active elements of the cancer stem cell niche.(45)

The role of chronic hyperglycemia in PSC activation has not been assessed prior to this patent application. Altogether three studies analysed the effects of high glucose concentrations not on human, but on rat PSC activation, however the longest of these studies lasted only for 3 days, that could not allow the assessment of the chronic effects, therefore these experiments might not be regarded as the model of the effects of diabetes mellitus on human PSCs. Furthermore, none of these studies has mentioned any relation even regarding rat pancreatic stellate cells between the hyperglycemia (even for short period, non-chronic) and the molecular targets identified in our patent application.(46-48)

### 1 c

### Chemokine (C-X-C motif) Ligand 12 (Stroma Derived Factor 1) and Insulin Like Growth Factor Binding Protein 2 in Tumor Development and in Pancreatic Cancer

In 2006 Ilona Kryczek and coworkers demonstrated that the chemokine ligand 12/stroma-derived factor (CXCL12/SDF-1, NCBI Gene ID: 6387.) multiplicatively participates in tumor pathogenesis.

They reported that:
1) CXCL12 promotes tumor growth.
2) CXCL12 enhances the vessel supply of the tumor (neovascularization).
3) CXCL12 contributes to immunosuppressive networks within the tumor microenvironment.
4) CXCL12 mediates tumor cell migration, adhesion, and invasion.
5) CXCL12 enhances metastasis formation

Therefore, authors suggested that the CXCL12 and its receptor CXCR4 are important targets in the development of novel anti-cancer therapies.(49)

Chemokines, including CXCL12 are small chemoattractant cytokine molecules that bind to specific G-protein coupled seven-span transmembrane receptors. Most chemokines bind to multiple receptors, and the chemokine CXCL12 binds to the receptors CXC receptor 4 (CXCR4, CD184) and CXC receptor 7.(50-54)

CXCR4 is a typical G-protein coupled receptor, the binding of CXCL12 to CXCR4 induces intracellular signaling through multiple pathways initiating signals related to chemotaxis, cell survival and/or proliferation, increase in intracellular calcium, and transcription of certain genes. CXCR4 is expressed on multiple cell types including lymphocytes, hematopoietic stein cells, endothelial and epithelial cells, and also cancer cells. The CXCR4 receptor is necessary for the vessel development (vascularization) of the gastrointestinal tract (that incorporates the pancreas as well).(55)

The CXCL12/CXCR4 axis is involved in tumor progression, angiogenesis, metastasis, and survival.(49, 56)

Although CXCR7 is phylogenetically closely related to chemokine receptors, it fails to couple to G-proteins. CXCR7 functions as a scavenger receptor for CXCL12 and both a critical function of the receptor in modulating the activity of the expressed CXCR4 in development and tumor formation, and intracellular signaling via CXCR4 independent pathways inducing intracellular signals (JAK-STAT) is suggested.(57)

High glucose activated the CXCL12-CXCR4-axis (signaling pathway) in vascular smooth muscle cells in autocrine manner, which enhanced the proliferation and chemotaxis of the cells.(58) In certain human cancers stromal fibroblasts promote tumor growth and angiogenesis through elevated CXCL12 secretion.(59)

CXCL12 was reported to recruit Treg cells and enhance the migration (chemotaxis) towards the tumor tissue, thus creating an immune-suppressive tumor-microenvironment.(60)

CXCR4 and CXCR7 are frequently co-expressed in human pancreatic cancer tissues and cell lines. It also has been described that Beta-arrestin-2 and K-Ras (Kirsten rat sarcoma viral oncogene homolog) dependent pathways coordinate the transduction of CXCL12 signals. It is an important observation that the knockdown of CXCR4 expression was able to decrease the levels of K-Ras activity. Based on these results the authors suggested that this pathway was identified as possible target for therapeutics, based on inhibiting CXCL12 intracellular signaling to halt the growth of pancreatic cancer (inhibition at the ligand level prevents signaling via both receptors).(61)

CXCR4 receptor is frequently expressed in metastatic pancreatic tumor cells and CXCR4 not only stimulates cell motility and invasion but also promotes cancer cell survival and proliferation.(62) Besides the high tumor grade, high CXCR4 expression was the strongest prognostic factor for distant recurrence in a recent study.(63)

Moreover it has been demonstrated that the majority of pancreatic cancer cell lineages (co)express CXCR4 and CXCR7(61) and that also PSC express CXCR4.(39) On the other hand, CXCL12 is not secreted by human pancreatic cancer cells, but secreted by PSCs.

The CXCL12 protein could be identified in PSC cell culture media and if pancreatic cancer cell line was treated with PSC-conditioned media it not only could promote the proliferation, migration and invasion of pancreatic cancer cells, but also these effects could be blocked by AMD3100, an inhibitor of CXCR4, one of Chemokine (C-X-C) Ligand (CXCL12, alias SDF-1) receptors.(39)

### 1 d, Insulin-Like Growth Factor (IGF)-Binding Proteins (IGFBPs)

### The Role of Insulin-Like Growth Factor Binding Protein-2 (IGFBP2, Gene ID: 3485)

Insulin-like growth factor (IGF)-binding proteins (IGFBPs) regulate the temporo-spatial availability of insulin-like growth factors (IGFs). Both stimulatory and inhibitory effects of IGFBPs on IGF actions were described, and IGFBPs have several IGF-independent effects. Aberrant expression of IGFBPs was described in several cancers.

### Insulin-Like Growth Factor Binding Protein-2 (IGFBP2, Gene ID: 3485) and Hyperglycemia, Diabetes Mellitus

Recently, Zhi and colleagues used 2D-liquid chromatography combined with mass spectrometry to identify changes in the serum in patients with type 1 diabetes mellitus (T1 DM) in comparison to healthy individuals.(64) IGFBP2 was increased nearly 5×-fold (4.87×-fold) in the serum of T1DM patients compared to healthy controls, even after correction for age, sex and genetic risk IGFBP2 and demonstrated the highest risk of having T1DM (OR=2.02) of all six candidate proteins analyzed in the study. Another study, two decades earlier showed a non-significant trend towards increased IGFBP2 levels in the serum of young T1DM patients. It was an interesting observation that untreated T1DM patients had significantly higher IGFBP2 levels than those T1 DM patients who were already treated with insulin.(65)

In healthy subjects postprandial fluctuations of insulin and glucose or glucose infusions do not result in significant changes of serum IGFBP2 concentrations. This suggests that acute fluctuations in glucose and insulin concentrations have no role in the alteration of IGFBP2 serum levels and this also supports that it is not possible to model the changes occurring in diabetes mellitus in acute, short duration (e.g. hyperglycemia induced by glucose infusion) time frame regarding neither the IGFBP2 concentrations.(66)

### Insulin-Like Growth Factor Binding Protein-2 and Pancreatic Cancer

Using isotope-code affinity tag (ICAT) technology and Tandem Mass Spectrometry (MS/MS), Chen and colleagues were able to perform the quantitative protein profiling of pancreatic cancer juice. The biological samples (pancreatic juice) were collected during ERCP (endoscopic-retrograde cholangio-pancreatography) and samples from patients with pancreatic adenocarcinoma were compared to the samples obtained from individuals with chronic pancreatitis or other benign pancreatic lesions or from those who were investigated with the suspicion of these (benign conditions).(67, 68) They demonstrated the increase of IGFBP2 (mean increase: 4.8-fold) levels in the pancreatic juice samples of pancreatic cancer patients compared to the normal pancreatic juice samples. The increase of IGFBP2 was validated by Western Blotting (WB), which demonstrated that IGFBP2 was not detectable in pancreatic juice from normal and pancreatitis patients, but it was detected in all pancreatic juices from pancreatic cancer patients. They also assessed pancreatic tissue samples using WB: IGFBP-2 was only marginally expressed in 25% of normal, 50% of pancreatitis and in contrast it was highly expressed in seven of eight (88%) of pancreatic cancer tissues.(67)

As concluded from the above it was not known from the current standing of the technology that diabetes mellitus and the secretion of CXCL12 and IGFBP2 by human pancreatic stellate cells are related.

The inventors of this patent discovered the above, and also recognized that the processes above are induced by hyperglycemia and in case of a pancreatic cancer in addition that these processes promote proliferation of tumor cells as feature of malignancy, (these processes) supress the immune response against the tumor cells, enhance the neovascularization of the tumor and increase the resistance of the tumor against chemo and radio therapy.

The inventors of this patent discovered that the chronic increase in glucose levels (chronic hyperglycemia) might have an important role in the development of pancreatic cancer and also that the development of pancreatic cancer due to chronic hyperglycemia or the growth, progression and metastasis formation of an already developed pancreatic cancer may be prevented/inhibited/delayed by the inhibition of CXCL12 and IGFBP2.

### DISCLOSURE OF INVENTION

According to this the subject of this invention is the application of Chemokine (C-X-C motif) Ligand 12 (CXCL12) and Insulin-Like Growth Factor Binding Protein 2 (IGFBP2) inhibitors in the treatment of pancreatic cancer with diabetes mellitus.

The expression “inhibition” in relation to the present invention should refer without limitation to a meaning for example as follows: the direct inhibition of CXCL12 and IGFBP2, the inhibition of CXCR4, the receptor of CXCL12, the inhibition of the CXCL12 signal transduction (postreceptor) pathways, including the inhibition of the PI3K (phosphoinositol 3-kinase), inhibition of FAK (Focal Adhesion Kinase), inhibition of SRC (v-src sarcoma (Schmidt-Ruppin A-2) viral oncogene homologue (avian)), inhibition of mitogen-activated protein kinase (MEK, MAPK), inhibition of extracellular signal regulated kinase 1 and 2 (ERK1/2), the inhibition of the CXCL12-CXCR7-JAK-STAT-NFKB signal transduction pathways, the inhibition of IGFBP2 by vaccination and all other methods that—for the expert—obviously result the inhibition of CXCL12 and IGFBP2.

Without limiting our invention to these inhibitors, the inhibitors in this invention may be as follows:

### CXCL12 Inhibitor:

### NOX-A12

### Manufacturer: Noxxon Pharma Ag

Target molecule: Chemokine (C-X-C motif) Ligand (CXCL12)

### Effect: Antagonist

Agent: 45-nucleotid length L-RNA oligonucleotide, connected to a 40 kDa polyethylene
glycol (PEG) molecule
Agent structure: spiegelmer

### A CXCL12 Inhibitors of CXCR4 (Receptor of CXCL12):

### 1) Plerixafor (AMD3100)

### Manufacturer: Genzyme Corporation

Alias: Mozobil, 110078-46-1, biciklam JM-2987, JM3100, SID791, 155148-31-5
Target molecule: type 4 C-X-C chemokine receptor (CXCR4)

### Effect: Antagonist

IUPAC name: 1,1′-[1,4-phenylenebis(methylene)]bis[1,4,8,11-tetraazacyclotetradecan]
Mode of delivery: subcutaneous injection
CAS number: 155148-31-5 33
ATC code: L03AX16

### PubChem: CID 65015

IUPHAR ligandum: 844

### DrugBank: DB06809

### 2) Anti-CXCR4 (BMS-936564/MDX-1338)

### Manufacturer: Bristol-Myers Squibb

Target molecule type 4 C-X-C chemokine receptor (CXCR4)

### Effect: Antagonist

Agent: entirely human monoclonal anti-human CXCR4 antibody

### IGFBP2-Vaccine:

DNA Plasmid Based Vaccine encoding the IGFBP2 amino acids 1-163
(pUMVC3-hIGFBP-2 multi-epitope plasmid DNA vaccine)

### Manufacturer: Fred Hutchinson Cancer Research Center/University of Washington Cancer Consortium

### IGFBP2—(RGD Domain Recognition) Receptors: Integrin Receptor Inhibitors

### MEDI-522 (Abergrin)

Humanized monoclonal antibody against human alpha V beta 3 integrin Manufacturer:

### MedImmune LLC

### Intetumumab (CNTO 95)

Humanized monoclonal antibody against human alpha V integrin subunit

### Manufacturer: Centocor, Inc.

### EMD525797

Chimera monoclonal antibody against human alpha V integrin subunit

### Manufacturer: Merck KGaA

### Cilengitide

Integrin inhibitor

### Manufacturer: Merck KGaA

### Additional Inhibitors of CXCL12 Signal Transduction (Postreceptor) Pathway:

### Inhibitors of CXCL12-CXCR4-PI3K-MAPK-ERR, and CXCL12-CXCR4-PI3K-FAK-SRC-ERK Pathways:

### PI3K (Phosphoinositol 3-Kinase) Inhibitors

The binding of CXCL12 to its receptor CXCR4 activates the PI3K in the cell in a G-protein dependent manner

### 1) BAY80-6946

### Manufacturer: Bayer 34

### 2) BKM120

### Manufacturer: ChemScene LLC

### 3) PX-866

### Manufacturer: Oncothyreon Inc

### FAK (Focal Adhesion Kinase) Inhibitors

### 1) GSK2256098

### Manufacturer: GlaxoSmithKline

### 2) PF-00562271

### Manufacturer: Pfizer (Verastem, Inc)

### 3) PF-04554878

### Manufacturer: Pfizer (Verastem, Inc.)

### 4): VS-4718,

### Manufacturer: Verastem, Inc

SRC (v-Src Sarcoma (Schmidt-Ruppin A-2) Viral Oncogene Homolog (Avian), Proto-Oncogene Tyrosine-Protein Kinase, Rous Sarcoma) Inhibitors

### 1) AZD0424

### Manufacturer: Astra Zeneca

### 2) Dasatinib (BMS-354825, Sprycel)

(oral multi-BCR/ABL és Src family tyrosin kinase inhibitor)
IUPAC name: N-(2-clorine-6-methylphenyl)-2-[[6-[4-(2-hydroxiethyl)-1-piperazinil]-2-methyl-4-pirimidinil]amino]-5-tiazol carboxamid monohydrate

### Manufacturer: Bistrol-Myers Squibb

### 3) KX2-391

### CAS No: 897016-82-9

### 4): Saracatinib (AZD0530)

### Manufacturer: Astra Zeneca

### Mitogen-Activated Protein Kinase (MEK, MAPK) Inhibitors

### 1) Inhibitor: ARRY-142886

### Manufacturer: Array BioPharma

### 2) BAY86-9766

### Manufacturer: Bayer

### 3) Trametinib (GSK1120212)

### Manufacturer: GlaxoSmithKiine

### 4) Selumetinib (AZD6244)

### Manufacturer: Astra Zeneca

### Extracellular-Signal-Regulated Kinase 1 és 2 (ERK1/2) Inhibitors

ERK is the last junction point in the MAPK pathway transcriptional programming

### 1) Inhibitor: SCH772984

### CAS No: 942183-80-4

Chemical name: (3R)-1-[2-oxo-2-[4-[4-(2-pyrimidinyl)-phenyl]-1-piperazinyl]ethyl]-N-[3-(4-pyridinyl)-1H-indazol-5-yl]-3-pirroliden-carboxamid

### 2) Inhibitor: BVD-523

### Manufacturer: BioMed Valley Discoveries, Inc

### CXCL12-CXCR7-JAK-STAT Signal Transduction Pathway Inhibitors:

### 1) Ruxolitinib

### Manufacturer Novartis, Incyte Corporation

### 2) SAR302503 (TG101348)

### Manufacturer: Sanofi

IUPAC name: N-tert-butyl-3-{5-methy-2-[4-(2-pyrrolidine-1-yl-ethoxi)-phenylamino]-pyrimidine-4-ylamino}-benzenesulfonamide

### CAS No: 936091-26-8

### 3) ISIS-STAT3Rx (ISIS 481464)

### Manufacturer: Isis Pharmaceuticals

### STAT3 Antisense Oligonucleotide Inhibitor

### 4) OPB-31121

### STAT3 Inhibitor

### Manufacturer: Otsuka Pharmaceutical Development & Commercialization, Inc.

### (and M.D. Anderson Cancer Center?)

### ClinicalTrials.gov Identifier: NCT00955812

In addition the subject of this invention is the production of the mentioned inhibitors of CXCL12 and IGFBP2 for the application of treatment of pancreatic cancer with diabetes mellitus.

### BEST MODE OF CARRYING OUT THE INVENTION

The subject of this invention also includes the drugs that contain the mentioned inhibitors of CXCL12 and IGFBP2 in combination with medically acceptable transfer, auxiliary or base vehicles.

The inhibitors in this invention may be produced by the traditional mixing, dissolving, granulating, tablet coating, grinding to wet powder, emulgeating, capsulation, incorporation or lyophilisation methods. The medicines may be formulated in a traditional method, with one or more physiologically acceptable vehicle, dilution substance or auxiliary substance that promote the production from inhibitors to a pharmacologically applicable preparations. The appropriate drug formulation depends on the delivery method selected by the professional/specialist or the individuals who is applying the treatment.

The inhibitors in this invention may be formulated for local administration as solutions, suspensions, etc that are well known from the literature.

The drug formulations intended for systemic administration includes those that are designed for use as injections, for example injections designed for subcutaneous, intravenous, intramuscular, intraperitoneal administration and also those that are designed for transdermal, transmucosal or oral administration.

The inhibitors in this invention may be formulated as injections that are appropriate for solutions, beneficial, physiologically compatible puffers, such as the Hank-solution, Ringer-solution or physiological saline solution. The solutions may contain formulating auxiliary substances, e.g.: suspending, stabilizing and/or dispersive substances.

The inhibitors in this invention may alternatively be administered in a form of a powder that is combined with an appropriate vehicle, such as sterile, pyrogen free water before use.

We use substances to promote penetration, according to the barrier in the formulations for transmucosal administration.

For the oral administration the inhibitors in this invention may be simply formulated by the combination of the inhibitors with the pharmacologically acceptable vehicles that are well known from the literature. These vehicles make possible the formulation of the inhibitors in this invention to tablets, pills, dragées, capsules, liquids, syrups, suspensions that are appropriate for oral delivery route (by mouth intake) for the treated patient. For the oral formulations, such as powders, capsules, tablets the appropriate additive vehicles include substances for example sugars, such as lactose, sacharose, mannitol and sorbitol, the cellulose preparations, e.g.: corn-starch, wheat-starch, rice-starch, potato-starch, gelatine, tagrakanta gum, methyl-cellulose, hydroxypropyl-methyl-cellulose, sodium-carboxy-methyl-cellulose, granulating substances and binding substances. We may add disintegrating substances, when it is needed, such as polyvinyl-pirrolidines, agar, or alginicacid, or their salts like sodium-alginate. We may add sugar coating or enterosolvent coating on the solid, uniformly dosed formulas when it is needed using the standard methods.

The water, glycols, oils, alcohols belong to the auxiliary vehicles, additives, dissolving substances appropriate for orally administered liquids, e.g: suspensions, elixirs, solutions. In addition, flavourings, preservatives, colouring substances may also be used.

The preparations intended for oral transmucosal (buccal) administration may be regularly formulated in tablet, sucking tablet, etc forms.

In addition to the previously mentioned drug formulations the inhibitors in this invention may be formulated as depot preparations. Such depot preparations may be administered via implantation (e.g.: subcutaneous implantation, or intramuscular implantation or bile duct and pancreatic drug eluting stent or also as an intramuscular injection). For the production of such depot preparations the inhibitors in this invention are used in an appropriate polymer or hydrophobic substances (for example as an emulsion in an acceptable oil) or with ion-changer resins or as weakly solving salts.

In addition we may use other drug-releasing pharmacological systems that are well known from the literature, such as liposomes, emulsions. We may also use organic solvents, e.g.: dimethyl-sulphoxide. The inhibitors in the invention may be used in extended-release systems, such as semi-permeable matrix of solid polymers containing the therapeutic drugs. Different materials providing extended drug release were produced and these are well known for the professional. The compounds, depending on the chemical structure of the extended drug release capsules, are released in a few weeks or more than 100 days.

Depending on the chemical structure and the biological stability of the therapeutic compounds further strategies may be used to stabilize the drugs, including pegylation, when a polyethylene-glycol (PEG) polymer chain is covalently bound to the drug molecule.

Drug-eluting bile duct and pancreatic stents may be used as additional drug-releasing systems, that release the inhibitor directly at the location where the tumor is occurred that provides a high anti-tumoral preventive/therapeutic efficacy. The placement of such stents to the appropriate location (e.g.: during endoscopic retrograde cholangiopancreatography) are well known for the professional.

### Methods

### 2 a) Pancreatic Stellate Cells

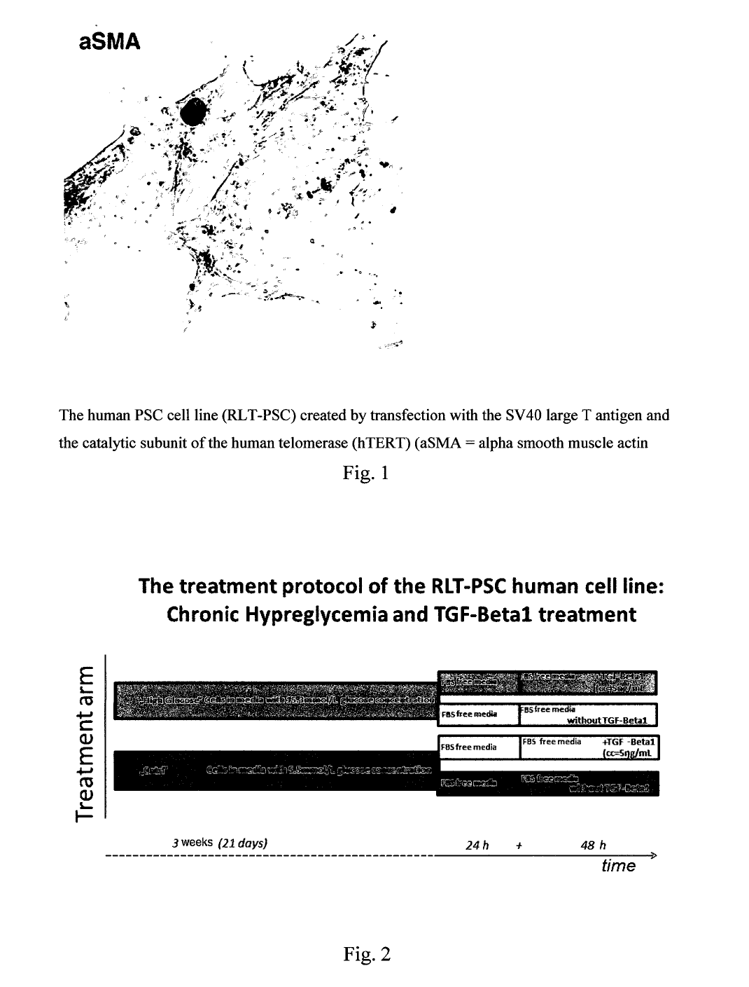

A human PSC line (RLT-PSC) was used for the experiments. PSCs isolated from a patient with chronic pancreatitis and immortalized by transfection with the SV40 large T antigen and the catalytic subunit of the human telomerase (hTERT) were used for the creation of the cell line.(17) (FIG. 1) The RLT-PSC cell lineage is an excellent tool for in vitro studies of the activation and the pathology of PSCs and to model pathologic processes leading to tissue fibrosis in the pancreas and it is also possible to study a pancreatic cancer-associated phenotype and secretion profile of PSCs using this cell line. FIG. 1 represents that the protein expression of alpha smooth muscle actin (aSMA) was detectable in nearly 100% of the cells of the RLT-PSC cell lineage.(17)

### 2 b) Cell Cultures

Cells were cultured at 37° C. atmosphere containing 5% CO2 and 100% humidity with Gibco® DMEM (Dulbecco's Modified Eagle Medium with 5.5 mmol/L glucose concentration, Life Technologies Corporation) containing 10% fetal bovine serum (FBS) and supplemented 100 U/mL penicillin, 100 microg/mL streptomycin and 1% L-Glutamine. Cells were passaged passages at 85-90% confluence using trypsin-EDTA. Cells were treated according to the following protocol:

### 2 c) Treatment Protocols

### Exposure to Chronic Hyperglycemia and Treatment with TGF-Beta1

The treatment protocol is indicated on the 2^{nd} figure (the treatment protocol of RLT-PSC cell lineage—exposure to chronic hyperglycemia and treatment with TGF-Beta1—on different treatment arms).

Cells on the control (Cntrl) arm were cultured in the conditions as described above using the Gibco® DMEM with a glucose concentration of 5.5 mmol/L.

Cells on the High glucose arm were cultured with Gibco® DMEM, High Glucose in 15.3 mmol/L glucose concentration. Cells were cultured for 3 weeks (21 days) on both arms due to that this time-frame is already appropriate for modeling diseases characterized by chronic hyperglycemia (diabetes mellitus, impaired fasting glucose levels, impaired glucose tolerance) and also due to that preliminary experiments showed best response in alteration of extracellular matrix (ECM) protein production with such a long time-frame. Subsequently, cells were cultured for 24 hours in FBS-free media and afterwards for 48 hours in a culture media supplemented either with or without TGF-Beta1 (cc=5 ng/mL). (FIG. 2) Four parallels wells were used for each regimen. After culturing, the cells were collected for RNA and protein analysis. For immunocytochemistry the cells were grown on Lab-Tek (Nunc GmbH & Co. KG Wiesbaden Germany) plates. Experiments were repeated three times.

### 3 Assessment of RLT-PSC Lineage Cultures after Different Treatments

### Assessment of Alterations in Gene Expression Profiles at mRNA Level

### 3 a) Gene Expression Chip (Array)

Forty-eight hours after stimulation with or without TGFB-1 RNA was isolated using the RNeasy Kit (Qiagen, Hilden, Germany) and the quantity was determined using the Gene Quant (Pharmacia) device. Integrity of the isolated. RNA was assessed using a BioRad Bioanalyzer, demonstrating a RIN above 7 (Mean RIN=9.2±SD 0.4) for all isolated RNA samples.

Two biological duplicates were pooled within each group and two technical duplicates were hybridized from each pooled sample group onto the GeneChip® PrimeView™ Human Gene Expression Array. Biotinylated aRNA probes were synthesized from 200 ng total RNA and fragmented using the 3′ IVT Express Kit according to the suggestions of the manufacturer (Affymetrix, Santa Clara, Calif., USA http://media.affymetrix.com/support/downloads/manuals/3_ivt_express_kit_manual.pdf). Ten ug of fragmented aRNA sample was hybridized into each of GeneChip® PrimeView™ Human Gene Expression Arrays (Affymetrix) for 16 hours at 45° C. and 60 rpm. Hybridized microarrays were washed and stained using antibody amplification staining method applying FS450_001 fluidics script and Fluidics Station 450 (Affymetrix) instrument subsequently, fluorescent signals were detected by GeneChip Scanner 3000 (Affymetrix) according to the manufacturer's instructions.

Data were extracted from the CEL files using “R” (software version 2.15) surface with Bioconductor software (version 2.11) packages. RMA normalization was performed and data were converted to Log 2 notation to make Feature selection by linear model and SAM (Significance analysis of Microarray) using “limma” and “samtools” packages. Gene (mRNA) expression values were ranked upon their differential expression compared to the samples isolated from the PSC cell cultures on the non-treated control arm that has been previously cultured for 3 weeks in normal (5.5 mmol/L) glucose concentration (controls).

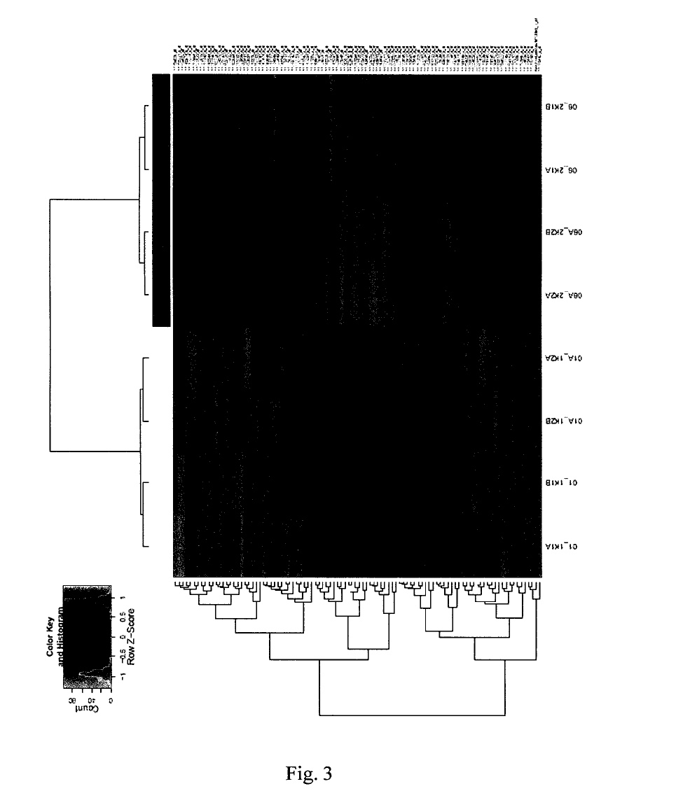

Two sets of genes were selected: one included 100 and the other one included 300 genes that provided the best separation of the control and the observed condition using a hierarchical clustering for visual demonstration—this is indicated in a heatmap for better visualization on FIG. 3. All top 300 (and 100) differentially expressed genes were significantly different from controls using a one-tailed Student-test on a Statistica software (version 10.0) and the p-value of 10⁻⁴, yet not all the fold-change expression values of differentially expressed genes reached the expression threshold suggested by the manufacturer.

In order to identify and rank important signal transduction pathways, networks, and potential disease associations the Kegg pathway and Wikipathways free databases were used. After ranking potentially altered pathways upon different treatments based on differential expression and also considering biological plausibility a set of differentially genes for further validation using the real-time RT PCR method was selected: DUSP1, DUSP10, TXNIP, CXCL12, DPP4, VCAN, FOS, LTBP2, EGR1, COL5a1, THBS1, PPARg, RND3, MMP1, BMP2, CTGF (we used the official gene name abbreviations that are available at the www.ncbi.nlm.nih.gov website). On FIG. 3 the heatmap of differentially expressed top 100 genes in PSCs with best separation of cells kept in normal glucose concentration or exposed to chronic hyperglycemia and no other treatment in order to model diabetes mellitus—a chronic disease. The explanation for the labels on the hetamap is as follows: from PSC samples of 01_1K1A-01_1K1B-01A_1K2B-01A_1K2A four parallel runs from normal (5.5 mmol/L glucose cc) and 06A_2K2A-06A_21(2B-06_2K1A-06_2K1B from high glucose (15.3 mmol/L) exposure treatment arms.

### 3 b) Real-Time RT-Polymerase Chain Reactions (Validation)

First strand cDNA was synthesized after DNase digestion with Deoxyribonuclease I-Amplification Grade (Sigma-Aldrich, St. Louis, Mo.) from 1 μg RNA using the SuperScript First-Strand Synthesis System for RT-PCR kit (Invitrogen, Karlsruhe, Germany) applying Oligo(dT) priming under the conditions recommended by the manufacturer. For each of the 16 genes, cDNA Real-time PCR assays were performed using Gene Expression Analysis with TaqMan® Assays in an ABI 7000 Sequence Detection System under conditions recommended by the manufacturer. Results were standardized to the 18S rRNA. Gene expression of each gene was recorded in 3 RT-PCR runs, and was first normalized against the reference gene based on the cycle threshold values (CT) as follows: ΔCT_{Examined gene}=CT_{Examined gene}−CT_{ref}, then the relative gene expression value was calculated as fold changes which is equal to the 2^{-ΔΔCT}, where ΔΔCT=ΔCT_{Observed sample}−ΔCT_{Control sample}.

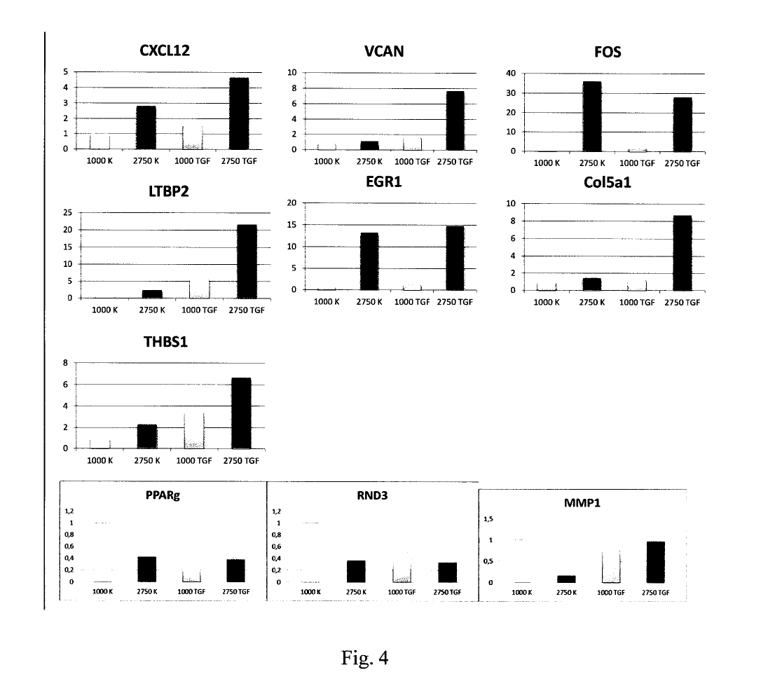

Control samples refer to samples as previously that were isolated from PSC cultures that were kept in 5.5 mmol/L glucose concentration and subsequently were not treated with growth factor (TGF-Beta1), control samples on the figures are indicated with “1000K” label. The mean fold changes of gene expressions at mRNA level of 10 selected genes are indicated on FIG. 4 (the alterations in the gene expressions of CXCL12 and DPP4 are indicated also in a separate section). The samples from different treatment arms are labeled as follows:

1000K=RNA samples isolated from PSCs were cultured in 5.5 mmol/L glucose concentration and subsequently were not treated with TGF-Beta1
2750K=exposure to chronic hyperglycemia (15.3 mmol/L—3 weeks) and no other treatment
1000 TGF=5.5 mmol/L glucose concentration and subsequent treatment with TGF-Beta1 (cc=5 ng/mL for 48 hours)
2750 TGF=exposure to chronic hyperglycemia (15.3 mmol/L—3 weeks) and subsequent treatment with TGF-Beta1 (cc=5 ng/mL for 48 hours)

### 3 c) Real-Time RT-PCR (Validation) of Change in CXCL12 Gene Expression at mRNA Level in PSCs Exposed to Chronic Hyperglycemia

Chemokine (C-X-C motif) ligand 12 mRNA expression was determined using the protocol and recommendations of the manufacturer (Applied Biosystems, TaqMan® Gene Expression Cat. #4331182 Assay for Human species) with FAM dye and an amplicon length of 77 bp. Results for CXCL12 mRNA expression using real-time RT PCR. The calculation of the results was done as described in section 3 b, and the results after different treatments of PSCs are indicated in table 2.

TABLE 2
Mean change in gene of CXCL12 expression at mRNA level (−fold) in
PSCs according to the treatment arm in human PSC (RLT-PSC) cell line.
Exposure to chronic hyperglycemia significantly* (p < 0.05 - using one
tailed Student test) upregulated CXCL12 mRNA expression in PSCs, both
when PSCs were subsequently remained untreated with any growth factor
(1000K vs 2750K) and also when PSCs were subsequently treated with
TGF-B1 (2750 TGF vs 1000 TGF).

| Treatment | Mean change in gene of CXCL12 | |
|---|---|---|
| Arm | expression at mRNA level (−fold) in PSCs | 95% CI |
| 1000K | 1.00E+00 | |
| 2750K | 2.36E+00* | 1.38 to 3.34 |
| 1000 TGF | 1.43E+00 | 0.94 to 1.93 |
| 2750 TGF | 4.02E+00* | 3.05 to 4.99 |

### Explanation of Labels:

1000K=RNA samples isolated from PSCs were cultured in 5.5 mmol/L glucose concentration and subsequently were not treated with TGF-Beta1
2750K=exposure to chronic hyperglycemia (15.3 mmol/L—3 weeks) and no other treatment
1000 TGF=5.5 mmol/L glucose concentration and subsequent treatment with TGF-Beta1 (cc=5 ng/mL for 48 hours)
2750 TGF=exposure to chronic hyperglycemia (15.3 mmol/L—3 weeks) and subsequent treatment with TGF-Beta1 (cc=5 ng/mL for 48 hours)

### 4 a) Identification of Glucose Transporters on Pancreatic Stellate Cells

Glucose transporters were not identified previously on pancreatic stellate cells. In order to identify which glucose transporters might be present on PSC Immunocytochemistry/Immunofluorescence assays were performed. Cells were fixed with methanol. After fixation, permeabilization and blocking nonspecific protein-protein interactions (2% BSA for 30 minutes at 22° C.) cells were incubated with the primary antibody overnight at +4° C.

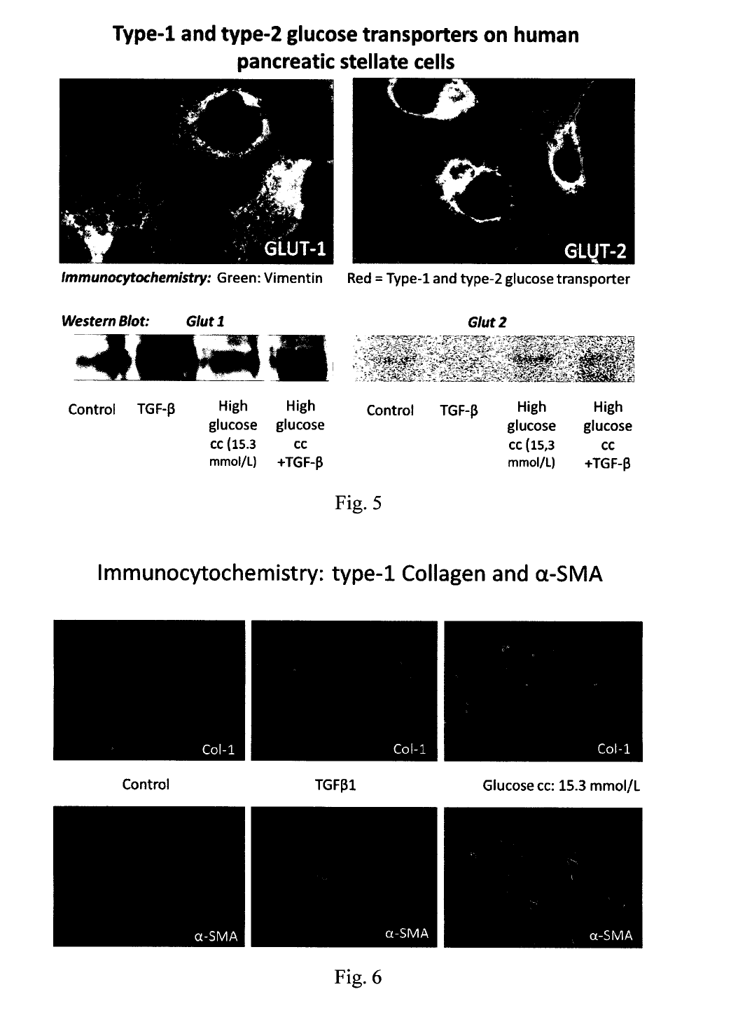

For secondary antibody polyclonal Goat anti-rabbit IgG (H+L) conjugated to Alexa Fluor 568 (red) at a 1/1000 dilution was used for 1 h. Cells were counterstained with DAPI (blue). (FIG. 5) FIG. 5 demonstrates the identification of type-1 and type-2 glucose transporters on human pancreatic stellate cells, on the RLT-PSC cell line using immunchytochemistry and Western Blot. We indicate the antibodies used in the experiments are indicated in table 3 below.

TABLE 3
Summary of different glucose transporter specific
antibodies used for the immunocytochemistry.

| Manufacturer | Cat No | Antibody specificity | Clonality | Isotype |
|---|---|---|---|---|
| Abcam, UK | AB652 | Anti-Glucose | Polyclonal | IgG |
| | | Transporter | | |
| | | GLUT1 antibody | | |
| Abcam, UK | AB54460 | Anti-Glucose | Polyclonal | IgG |
| | | Transporter | | |
| | | GLUT2 antibody | | |
| Abcam, UK | AB41525 | Anti-Glucose | Polyclonal | IgG |
| | | Transporter | | |
| | | GLUT3 antibody | | |
| Abcam, UK | AB654 | Anti-Glucose | Polyclonal | IgG |
| | | Transporter | | |
| | | GLUT4 antibody | | |

(Cat = catalogue)

### 4 b) Activation (Trans-Differentiation) and Collagen-1 Production of Pancreatic Stellate Cells after Exposure to Chronic Hyperglycemia

In order to prove that PSCs undergo trans-differentiation (activation) due to chronic hyperglycemia exposure, alpha-Smooth Muscle Actin (α-SMA) fibrillary structures in the cytoplasm have been assessed. In addition—as activated PSCs that are the major source of Extracellular Matrix Proteins (ECM) in different diseases, like Collagens type 1 and 3 in the pancreas, also intracellular Collagen-1 was assessed using Immunocytochemistry. Cells were fixed with methanol and underwent the protocol described in section 4 a, using the primary antibodies as indicated in table 4. and the result of such a representative experiment is demonstrated on FIG. 6. FIG. 6. indicates the activation of pancreatic stellate cells and the increase in the production of type-1 Collagen upon exposure to chronic hyperglycemia or TGF-Beta1 treatment.

TABLE 4
Antibodies used for the assessment of PSC activation
(trans-differentiation) and ECM production

| Manufacturer | Cat No | Antibody specificity | Clonality | Isotype |
|---|---|---|---|---|
| Abcam, UK | AB34710 | Anti-Collagen I | Polyclonal | IgG |
| | | antibody | | |
| Epitomics | 5264 | Alpha-Actin | Monoclonal | IgG |
| (AbCam) | | (Smooth Muscle) | | |
| | | (ACTA2) antibody | | |

Pancreatic stellate cells are imagined on FIG. 6.: cells that were kept in media with normal glucose concentration for 3 weeks (2 photos on the left side—untreated ‘control’ cells) or subsequently treated with TGF-Beta1 (concentration=5 ng/mL) for 48 h (2 photos on the middle—“TGFβ1”) and cells that were exposed to hyperglycemia for 3 weeks (2 photos on the right—glucose concentration: 15.3 mmol/L). The experiment was performed using pancreatic stellate cells of the human pancreatic stellate cell line (RLT-PSC) that was created from human pancreas and immortalized by transfection with the SV40 large T antigen and the catalytic subunit of the human telomerase (hTERT).(17) The increase in the amount of intracytolpasmic alpha-Smooth Muscle Actin (α-SMA) could be observed—typically forming fibrillary structures using immunocytochemistry and increase in the amount of type-1 Collagen-1 could be observed in activated state as a response to chronic hyperglycemia exposure. The contribution of the growth factor, TGF-Beta-1 to the activation process was previously known.

### 5 a) Protein Level Validation of Target Molecules Identified by the Exposure of Pancreatic Stellate Cells to Chronic Hyperglycemia

### CXCL12

The amount of human CXCL12 protein was measured in three repeated biological samples, at each measurement with technical duplicates using a Solid Phase Sandwich ELISA and 10 uL culture supernatant per well (Human Quantikine ELISA Kit, R&D System, Cat No: DSA00) using conditions as suggested by the manufacturer (R² value of the standard curve using solutions provided by the manufacturer with standard (known) CXCL12 concentration was: 0.9983). The amount of human CXCL12 protein secreted by PSCs are indicated in table 5—according to treatment arms. The validation of the quantitative changes of the identified target molecule, CXCL12 using ELISA measurement is indicated in table 5. Human Pancreatic Stellate Cells increased their CXCL12 secretion* after exposure to chronic hyperglycemia (glucose concentration: 15.3 mmol/L—for 3 weeks)

TABLE 5

| | Mean (fold) | Mean CXCL12 | | |
|---|---|---|---|---|
| | change in CXCL12 | concentration | | |
| | concentration in | (pg/mL) in | | |
| | the supernatant | the supernatant | | |
| | of PSC cultures | of PSC cultures | 95% CI | 95% CI |
| Treatment | after different | after different | lower | upper |
| Arm | treatments | treatments | value | value |
| 1000K | 1 | 309.75 | 271.39 | 348.11 |
| 1000 TGF | 1.89 | 586 | 99.12 | 1071.74 |
| 2750K | 2.22* | 688.22 | 368.60 | 1006.69 |
| 2750 TGF | 2.61* | 809.75 | 579.14 | 1037.67 |

Changes * marked are significant using one-way ANOVA

### IGFBP2

The amount of human IGFBP-2 protein was measured in three repeated biological samples, at each measurement with technical duplicates using a Solid Phase Sandwich ELISA and 50 uL culture supernatant per well (Human Quantikine ELISA Kit, R&D System, Cat No: DGB200) according to the recommendations of the manufacturer (R² value of the standard curve using solutions provided by the manufacturer with standard (known) IGFBP-2 concentration was: 0.964.) The amount of human IGFBP2 protein secreted by PSCs are indicated in table 6—according to treatment arms.

TABLE 6
The validation at protein level of the quantitative changes
of the identified target molecule, IGFBP2 using ELISA measurement.
Human Pancreatic Stellate Cells increased their IGFBP2 secretion*
after exposure to chronic hyperglycemia (glucose concentration:
15.3 mmol/L - for 3 weeks)

| | Mean (fold) | Mean IGFBP-2 | | |
|---|---|---|---|---|
| | change in IGFBP-2 | concentration | 95% CI | 95% CI |
| | concentration in | (ng/mL) in | lower | upper |
| | the supernatant | the supernatant | value | value |
| | of PSC cultures | of PSC cultures | (IGFBP-2 | (IGFBP-2 |
| Treatment | after different | after different | concen- | concen- |
| Arm | treatments | treatments | tration) | tration) |
| 1000K | 1 | 0.73 | 0.46 | 1 |
| 1000 TGF | 2.08 | 1.51 | 0.81 | 2.26 |
| 2750K | 3.48* | 2.53 | 0.91 | 4.18 |
| 2750 TGF | 4.89* | 3.55 | 2.71 | 4.8 |

Changes * marked are significant using one-way ANOVA

We used the following labels to indicate samples of PSCs from different treatment arms in table 5 and 6.

1000K=control samples isolated from PSCs were cultured in 5.5 mmol/L glucose concentration for 3 weeks and subsequently were not treated with TGF-Beta1
1000 TGF=samples from PSCs cultured in 5.5 mmol/L glucose concentration for 3 weeks and subsequent treatment with TGF-Beta1 (cc=5 ng/mL for 48 hours)
2750K=samples from PSCs exposed to chronic hyperglycemia (15.3 mmol/L—3 weeks), but no other treatment
2750 TGF=samples from PSCs exposed to chronic hyperglycemia (15.3 mmol/L—3 weeks) and subsequent treatment with TGF-Beta1 (cc=5 ng/mL for 48 hours)

### 6. Dipeptidyl-Peptidase 4 (DPP4, Gene ID: 1803) and the Pancreatic Stellate Cell Line Exposed to Different Treatments

Dipeptidyl-peptidase 4 (DPP4, Gene ID: 1803) protein has two forms: a membrane bound and a soluble form. The enzymatic activity of DPP4 is exerted in dimerized form when it cleaves 2 amino acids at the NH2-terminal end from a number of protein molecules with important biological functions, including CXCL12. A number of proteins with important biological functions e.g.: incretin hormones or CXCL10 (69-71) loose of their biological activity as a consequence of DPP4 processing (cleavage of NH2-terminal residues). Therefore it was highly important to assess that the treatments that result in altered mRNA expression and protein level of CXCL12 alteration of the DPP4 mRNA expression would also have an impact on the DPP4 in the mRNA expression array or the DPP4 enzymatic activity in the cell culture media.

### Methods and Results:

The DPP4 mRNA expression was calculated from the expression array, the results as indicated table 7 as follows:

TABLE 7

| Treatment | Mean fold change of the DPP4 gene expression at mRNA |
|---|---|
| arm | level in PSCs using expression array (−fold change) |
| 1000K | 1 |
| 1000 TGF-B | 0.906 |
| 2750K | 0.725 |
| 2750 TGF-B | 0.969 |

Subsequently the gene expression of DPP4 at mRNA level was also validated in a Real-time RT-Polymerase Chain Reaction (as described in section 3b) using a TAQMan (ABI, Cat. #4331182) assay as suggested by the manufacturer. Results are indicated in the table 8. as follows:

TABLE 8

| Treatment | Mean fold change of the DPP4 gene expression in at mRNA |
|---|---|
| arm | level in PSCs using real-time RT_PCR (−fold change) |
| 1000K | 1 |
| 1000 TGF | 1.089 |
| 2750K | 0.516 |
| 2750 TGF | 0.457 |

We used the following labels to indicate samples of PSCs from different treatment arms in table 7 and 8.

1000K=control samples isolated from PSCs were cultured in 5.5 mmol/L glucose concentration for 3 weeks and subsequently were not treated with TGF-Beta1
1000 TGF=samples from PSCs cultured in 5.5 mmol/L glucose concentration for 3 weeks and subsequent treatment with TGF-Beta1 (cc=5 ng/mL for 48 hours)
2750K=samples from PSCs exposed to chronic hyperglycemia (15.3 mmol/L—3 weeks), but no other treatment
2750 TGF=samples from PSCs exposed to chronic hyperglycemia (15.3 mmol/L—3 weeks) and subsequent treatment with TGF-Beta1 (cc=5 ng/mL for 48 hours)

### Measurement of DPP4 Enzymatic Activity in PSC Culture Supernatant

The DPP4 enzymatic activity was measured in the supernatant of cultured human immortalized PSC cell lineage on all different treatment arms and on the control arm. The measurements were carried out at 37° C. in continuous monitoring microplate (Corning) based kinetic assay on Varioskan Flash (Thermo Scientific, USA) reader. 100 uL PSC supernatant was removed the reaction was done in a 125 uL total reaction volume with the Tris-HCL (100 mM, pH: 7.6) buffer and the 1-1-Gly-Pro-pNA*p-tosylate (Bachem, Bubendorf, Switzerland, Cat No.: L-1295 0100) that was used as substrate in 3 mM final concentration. The increase of the UV absorption at 405 nm (OD405) caused by the DPP4-proteolytic release of p-nitroanilide from GlyPro-p-nitroanilide was continuously monitored for 30 minutes. The OD405 values of the reaction mixtures before the addition of GlyPro-pnitroanilide were subtracted from the obtained values at 30′ minutes and also the mean of the OD405 values of two blank runs (runs without PSC supernatant) were also subtracted to represent the real increase of OD405 values as a measurement of proteolytic activity. Results were expressed in unit per liter (U/L) after factor calculations. The DPP4 enzymatic activity values are indicated in table 9.

TABLE 9

| Treatment | Mean DPP4 Enzymatic Activity (U/L) in PSC |
|---|---|
| arm | supernatant according to different treatments |
| 1000K | 23.26 |
| 1000 TGF | 23.08 |
| 2750K | 21.87 |
| 2750 TGF | 23.16 |

We used the following labels to indicate samples of PSCs from different treatment arms in table 9.

1000K=control samples isolated from PSCs were cultured in 5.5 mmol/L glucose concentration for 3 weeks and subsequently were not treated with TGF-Beta1
1000 TGF=samples from PSCs cultured in 5.5 mmol/L glucose concentration for 3 weeks and subsequent treatment with TGF-Beta1 (cc=5 ng/mL for 48 hours)
2750K=samples from PSCs exposed to chronic hyperglycemia (15.3 mmol/L—3 weeks), but no other treatment
2750 TGF=samples from PSCs exposed to chronic hyperglycemia (15.3 mmol/L—3 weeks) and subsequent treatment with TGF-Beta1 (cc=5 ng/mL for 48 hours)
Interpretation of the Results Obtained from DPP4 mRNA Expression and Enzymatic Activity Measurements:

The DPP4 mRNA expression was down-regulated after exposure to chronic hyperglycemia in pancreatic stellate cells according to real-time RT-PCR results, however these alterations were only observed as trends in the expression array. In the supernatant of the cultured PSCs no significant changes were observed in the DPP4 enzymatic activity after exposure to chronic hyperglycemia.

Therefore the increase of CXCL12 protein level in the in the supernatant of PSCs exposed to chronic hyperglycemia was not followed by the increase of DPP4 enzymatic activity that cleaves 2 amino acids at the N-terminal end of the CXCL12 molecule. In contrast the DPP4 gene expression at mRNA level was rather down-regulated. The experiments demonstrate that the cleavage of CXCL12 by DPP4 certainly not increased, therefore the excess CXCL12 protein occurring in the cell culture media of PSCs as a result of exposure to chronic hyperglycemia is not accompanied by an increased degradation by the DPP4 enzyme.

Collectively, it was not known according to the current standing of the technics that diabetes mellitus and increased secretion of CXCL12 and IGFBP2 by human pancreatic stellate cells are related. The processes above induced by hyperglycemia, a characteristics of diabetes mellitus, in addition that have an effect on the biological characteristics determining the malignant features of pancreatic tumor cells, promote their proliferation weaken the immune response against the tumor cells, furthermore promote vascularization and induce the resistance of the tumor against chemo and radiotherapy. Therefore this invention overcomes a serious prejudice due to that this process—according to the current standing of the technics—has not been related to diabetes mellitus that is characterized by chronically higher than normal glucose levels.

### REFERENCES

1. Ben Q, Xu M, Ning X, Liu J, Hong S, Huang W, et al. Diabetes mellitus and risk of pancreatic cancer: A meta-analysis of cohort studies. Eur J Cancer. 2011; 47(13):1928-37.
2. Ben Q, Cai Q, Li Z, Yuan Y, Ning X, Deng S, et al. The relationship between new-onset diabetes mellitus and pancreatic cancer risk: a case-control study. Eur J Cancer. 2011; 47(2):248-54.
3. Chari S T, Leibson C L, Rabe K G, Ransom J, de Andrade M, Petersen G M. Probability of pancreatic cancer following diabetes: a population-based study. Gastroenterology. 2005; 129(2):504-11.
4. Huxley R, Ansary-Moghaddam A, Berrington de Gonzalez A, Barzi F, Woodward M. Type-II diabetes and pancreatic cancer: a meta-analysis of 36 studies. Br J Cancer. 2005; 92(11):2076-83.
5. Li C, Balluz L S, Ford E S, Okoro C A, Tsai J, Zhao G. Association between diagnosed diabetes and self-reported cancer among U.S. adults: findings from the 2009 Behavioral Risk Factor Surveillance System. Diabetes Care. 2011; 34(6):1365-8.
6. Stevens R J, Roddam A W, Beral V. Pancreatic cancer in type 1 and young-onset diabetes: systematic review and meta-analysis. Br J Cancer. 2007; 96(3):507-9.
7. Perrin M C, Terry M B, Kleinhaus K, Deutsch L, Yanetz R, Tiram E, et al. Gestational diabetes as a risk factor for pancreatic cancer: a prospective cohort study. BMC Med. 2007; 5:25.
8. Sella T, Chodick G, Barchana M, Heymann A D, Porath A, Kokia E, et al. Gestational diabetes and risk of incident primary cancer: a large historical cohort study in Israel. Cancer Causes Control. 2011; 22(11):1513-20.
9. Jee S H, Ohrr H, Sull J W, Yun J E, Ji M, Samet J M. Fasting serum glucose level and cancer risk in Korean men and women. JAMA. 2005; 293(2):194-202.
10. Society A C. Cancer Facts & Figures 2013. Atlanta: American Cancer Society; 2013. 2013.
11. Bachem M G, Schneider E, Gross H, Weidenbach H, Schmid R M, Menke A, et al. Identification, culture, and characterization of pancreatic stellate cells in rats and humans. Gastroenterology. 1998; 115(2):421-32.
12. Apte M V, Haber P S, Applegate T L, Norton I D, McCaughan G W, Korsten M A, et al. Periacinar stellate shaped cells in rat pancreas: identification, isolation, and culture. Gut. 1998; 43(1):128-33.
13. Erkan M, Adler G, Apte M V, Bachem M G, Buchholz M, Detlefsen S, et al. StellaTUM: current consensus and discussion on pancreatic stellate cell research. Gut. 2012; 61(2):172-8.
14. Kimura W, Kuroda A, Makuuchi M. Problems in the diagnosis and treatment of a so-called mucin-producing tumor of the pancreas. Pancreas. 1998; 16(3):363-9.
15. Fujita H, Ohuchida K, Mizumoto K, Nakata K, Yu J, Kayashima T, et al. alpha-Smooth Muscle Actin Expressing Stroma Promotes an Aggressive Tumor Biology in Pancreatic Ductal Adenocarcinoma. Pancreas. 2010.
16. Schreiber H, Rowley D A. Cancer. Awakening immunity. Science. 2010; 330(6005):761-2.
17. Jesnowski R, Furst D, Ringel J, Chen Y, Schrodel A, Kleeff J, et al. Immortalization of pancreatic stellate cells as an in vitro model of pancreatic fibrosis: deactivation is induced by matrigel and N-acetylcysteine. Lab Invest. 2005; 85(10):1276-91.
18. Apte M V, Pirola R C, Wilson J S. Pancreatic stellate cells: a starring role in normal and diseased pancreas. Front Physiol. 2012; 3:344.
19. Apte M V, Haber P S, Darby S J, Rodgers S C, McCaughan G W, Korsten M A, et al. Pancreatic stellate cells are activated by proinflammatory cytokines: implications for pancreatic fibrogenesis. Gut. 1999; 44(4):534-41.
20. Schneider E, Schmid-Kotsas A, Zhao J, Weidenbach H, Schmid R M, Menke A, et al. Identification of mediators stimulating proliferation and matrix synthesis of rat pancreatic stellate cells. Am J Physiol Cell Physiol. 2001; 281(2):C532-43.
21. Ohnishi N, Miyata T, Ohnishi H, Yasuda H, Tamada K, Ueda N, et al. Activin A is an autocrine activator of rat pancreatic stellate cells: potential therapeutic role of follistatin for pancreatic fibrosis. Gut. 2003; 52(10):1487-93.
22. Mews P, Phillips P, Fahmy R, Korsten M, Pirola R, Wilson J, et al. Pancreatic stellate cells respond to inflammatory cytokines: potential role in chronic pancreatitis. Gut. 2002; 50(4):535-41.
23. Tahara H, Sato K, Yamazaki Y, Ohyama T, Horiguchi N, Hashizume H, et al. Transforming growth factor-alpha activates pancreatic stellate cells and may be involved in matrix metalloproteinase-1 upregulation. Lab Invest. 2013; 93(6):720-32.
24. Charo C, Holla V, Arumugam T, Hwang R, Yang P, Dubois R N, et al. Prostaglandin E2 regulates pancreatic stellate cell activity via the EP4 receptor. Pancreas. 2013; 42(3):467-74.
25. Berna M J, Seiz O, Nast J F, Benten D, Blaker M, Koch J, et al. CCK1 and CCK2 receptors are expressed on pancreatic stellate cells and induce collagen production. The Journal of biological chemistry. 2010; 285(50):38905-14.
26. Fitzner B, Walzel H, Sparmann G, Emmrich J, Liebe S, Jaster R. Galectin-1 is an inductor of pancreatic stellate cell activation. Cell Signal. 2005; 17(10):1240-7.
27. Apte M V, Phillips P A, Fahmy R G, Darby S I, Rodgers S C, McCaughan G W, et al. Does alcohol directly stimulate pancreatic fibrogenesis? Studies with rat pancreatic stellate cells. Gastroenterology. 2000; 118(4):780-94.
28. Hu R, Wang Y L, Edderkaoui M, Lugea A, Apte M V, Pandol S J. Ethanol augments PDGF-induced NADPH oxidase activity and proliferation in rat pancreatic stellate cells. Pancreatology. 2007; 7(4):332-40.
29. Masamune A, Satoh A, Watanabe T, Kikuta K, Satoh M, Suzuki N, et al. Effects of ethanol and its metabolites on human pancreatic stellate cells. Dig Dis Sci. 2010; 55(1):204-11.
30. Asaumi H, Watanabe S, Taguchi M, Tashiro M, Otsuki M. Externally applied pressure activates pancreatic stellate cells through the generation of intracellular reactive oxygen species. Am J Physiol Gastrointest Liver Physiol. 2007; 293(5):G972-8.
31. Masamune A, Watanabe T, Kikuta K, Satoh K, Shimosegawa T. NADPH oxidase plays a crucial role in the activation of pancreatic stellate cells. Am J Physiol Gastrointest Liver Physiol. 2008; 294(1):G99-G108.
32. Kikuta K, Masamune A, Watanabe T, Ariga H, Itoh H, Hamada S, et al. Pancreatic stellate cells promote epithelial-mesenchymal transition in pancreatic cancer cells. Biochem Biophys Res Commun 2010; 403(3-4):380-4.
33. Kraman M, Bambrough P J, Arnold J N, Roberts E W, Magiera L, Jones J O, et al. Suppression of antitumor immunity by stromal cells expressing fibroblast activation protein-alpha. Science. 2010; 330(6005):827-30.
34. Löhr J M, Jesnowski R. Pancreatic stellate cells and pancreatic carcinoma: an unholy alliance. JOP. 2009; 10(4):472-3.
35. Vonlaufen A, Joshi S, Qu C, Phillips P A, Xu Z, Parker N R, et al. Pancreatic stellate cells: partners in crime with pancreatic cancer cells. Cancer research. 2008; 68(7):2085-93.
36. Bachem M G, Zhou S, Buck K, Schneiderhan W, Siech M. Pancreatic stellate cells—role in pancreas cancer. Langenbecks Arch Surg. 2008; 393(6):891-900.
37. Xu Z, Vonlaufen A, Phillips P A, Fiala-Beer E, Zhang X, Yang L, et al. Role of pancreatic stellate cells in pancreatic cancer metastasis. Am J Pathol. 2010; 177(5):2585-96.
38. Fujita H, Ohuchida K, Mizumoto K, Egami T, Miyoshi K, Moriyama T, et al. Tumor-stromal interactions with direct cell contacts enhance proliferation of human pancreatic carcinoma cells. Cancer Sci. 2009; 100(12):2309-17.
39. Gao Z, Wang X, Wu K, Zhao Y, Hu G. Pancreatic stellate cells increase the invasion of human pancreatic cancer cells through the stromal cell-derived factor-1/CXCR4 axis. Pancreatology. 2010; 10(2-3):186-93.
40. Hwang R E, Moore T, Arumugam T, Ramachandran V, Amos K D, Rivera A, et al. Cancer-associated stromal fibroblasts promote pancreatic tumor progression. Cancer research. 2008; 68(3):918-26.
41. Brittan M, Wright N A. Gastrointestinal stem cells. J Pathol. 2002; 197(4):492-509.
42. Magee J A, Piskounova E, Morrison S J. Cancer stem cells: impact, heterogeneity, and uncertainty. Cancer Cell. 2012; 21(3):283-96.
43. Cho R W, Clarke M F. Recent advances in cancer stem cells. Curr Opin Genet Dev. 2008; 18(1):48-53.
44. Li C, Heidt D G, Dalerba P, Burant C F, Zhang L, Adsay V, et al. Identification of pancreatic cancer stem cells. Cancer research. 2007; 67(3):1030-7.
45. Hamada S, Masamune A, Takikawa T, Suzuki N, Kikuta K, Hirota M, et al. Pancreatic stellate cells enhance stein cell-like phenotypes in pancreatic cancer cells. Biochem Biophys Res Commun 2012; 421(2):349-54.
46. Hong O K, Lee S H, Rhee M, Ko S H, Cho J H, Choi Y H, et al. Hyperglycemia and hyperinsulinemia have additive effects on activation and proliferation of pancreatic stellate cells: possible explanation of islet-specific fibrosis in type 2 diabetes mellitus. Journal of cellular biochemistry. 2007; 101(3):665-75.
47. Nomiyama Y, Tashiro M, Yamaguchi T, Watanabe S, Taguchi M, Asaumi H, et al. High glucose activates rat pancreatic stellate cells through protein kinase C and p38 mitogen-activated protein kinase pathway. Pancreas. 2007; 34(3):364-72.
48. Ko S H, Hong O K, Kim J W, Ahn Y B, Song K H, Cha B Y, et al. High glucose increases extracellular matrix production in pancreatic stellate cells by activating the renin-angiotensin system. Journal of cellular biochemistry. 2006; 98(2):343-55.
49. Kryczek I, Wei S, Keller E, Liu R, Zou W. Stroma-derived factor (SDF-1/CXCL12) and human tumor pathogenesis. Am J Physiol Cell Physiol. 2007; 292(3):C987-95.
50. Balkwill F. Cancer and the chemokine network. Nat Rev Cancer. 2004; 4(7):540-50.
51. Murdoch C. CXCR4: chemokine receptor extraordinaire. Immunol Rev. 2000; 177:175-84.
52. Nagasawa T, Tachibana K, Kishimoto T. A novel CXC chemokine PBSF/SDF-1 and its receptor CXCR4: their functions in development, hematopoiesis and HIV infection. Semin Immunol. 1998; 10(3):179-85.
53. Miao Z, Luker K E, Summers B C, Berahovich R, Bhojani M S, Rehemtulla A, et al. CXCR7 (RDC1) promotes breast and lung tumor growth in vivo and is expressed on tumor-associated vasculature. Proc Natl Acad Sci USA. 2007; 104(40):15735-40.
54. Balabanian K, Lagane B, Infantino S, Chow K Y, Harriague J, Moepps B, et al. The chemokine SDF-1/CXCL12 binds to and signals through the orphan receptor RDC1 in T lymphocytes. The Journal of biological chemistry. 2005; 280(42):35760-6.
55. Tachibana K, Hirota S, Iizasa H, Yoshida H, Kawabata K, Kataoka Y, et al. The chemokine receptor CXCR4 is essential for vascularization of the gastrointestinal tract. Nature. 1998; 393(6685):591-4.
56. Teicher B A, Fricker S P. CXCL12 (SDF-1)/CXCR4 pathway in cancer. Clin Cancer Res. 2010; 16(11):2927-31.
57. Naumann U, Cameroni E, Pruenster M, Mahabaleshwar H, Raz E, Zerwes H G, et al. CXCR7 functions as a scavenger for CXCL12 and CXCL11. PloS one. 2010; 5(2):e9175.
58. Jie W, Wang X, Zhang Y, Guo J, Kuang D, Zhu P, et al. SDF-1alpha/CXCR4 axis is involved in glucose-potentiated proliferation and chemotaxis in rat vascular smooth muscle cells. International journal of experimental pathology. 2010; 91(5):436-44.
59. Orimo A, Gupta P B, Sgroi D C, Arenzana-Seisdedos F, Delaunay T, Naeem R, et al. Stromal fibroblasts present in invasive human breast carcinomas promote tumor growth and angiogenesis through elevated SDF-1/CXCL12 secretion. Cell. 2005; 121(3):335-48.
60. Durr C, Pfeifer D, Claus R, Schmitt-Graeff A, Gerlach U V, Graeser R, et al. CXCL12 mediates immunosuppression in the lymphoma microenvironment after allogeneic transplantation of hematopoietic cells. Cancer research. 2010; 70(24):10170-81.
61. Heinrich E L, Lee W, Lu J, Lowy A M, Kim J. Chemokine CXCL12 activates dual CXCR4 and CXCR7-mediated signaling pathways in pancreatic cancer cells. J Transl Med. 2012; 10:68.
62. Marchesi F, Monti P, Leone B E, Zerbi A, Vecchi A, Piemonti L, et al. Increased survival, proliferation, and migration in metastatic human pancreatic tumor cells expressing functional CXCR4. Cancer research. 2004; 64(22):8420-7.
63. Bachet J B, Marechal R, Demetter P, Bonnetain F, Couvelard A, Svrcek M, et al. Contribution of CXCR4 and SMAD4 in predicting disease progression pattern and benefit from adjuvant chemotherapy in resected pancreatic adenocarcinoma. Ann Oncol. 2012; 23(9):2327-35.
64. Zhi W, Sharma A, Purohit S, Miller E, Bode B, Anderson S W, et al. Discovery and validation of serum protein changes in type 1 diabetes patients using high throughput two dimensional liquid chromatography-mass spectrometry and immunoassays. Molecular & cellular proteomics: MCP. 2011; 10(11):M111 012203.
65. Strasser-Vogel B, Blum W F, Past R, Kessler U, Hoeflich A, Meiler B, et al. Insulin-like growth factor (IGF)-I and -II and IGF-binding proteins-1, -2, and -3 in children and adolescents with diabetes mellitus: correlation with metabolic control and height attainment. The Journal of clinical endocrinology and metabolism. 1995; 80(4):1207-13.
66. Clemmons D R, Snyder D K, Busby W H, Jr. Variables controlling the secretion of insulin-like growth factor binding protein-2 in normal human subjects. The Journal of clinical endocrinology and metabolism. 1991; 73(4):727-33.
67. Chen R, Pan S, Yi E C, Donohoe S, Bronner M P, Potter J D, et al. Quantitative proteomic profiling of pancreatic cancer juice. Proteomics. 2006; 6(13):3871-9.
68. Chen R, Brentnall T A, Pan S, Cooke K, Moyes K W, Lane Z, et al. Quantitative proteomics analysis reveals that proteins differentially expressed in chronic pancreatitis are also frequently involved in pancreatic cancer. Molecular & cellular proteomics: MCP. 2007; 6(8):1331-42.
69. Proost P, Struyf S, Loos T, Gouwy M, Schutyser E, Conings R, et al. Coexpression and interaction of CXCL10 and CD26 in mesenchymal cells by synergising inflammatory cytokines: CXCL8 and CXCL10 are discriminative markers for autoimmune arthropathies. Arthritis Res Ther. 2006; 8(4):R107.
70. Nauck M, Smith U. Incretin-based therapy: how do incretin mimetics and DPP-4 inhibitors fit into treatment algorithms for type 2 diabetic patients? Best Pract Res Clin Endocrinol Metab. 2009; 23(4):513-23.
71. Baggio L L, Drucker D J. Biology of incretins: GLP-1 and GIP. Gastroenterology. 2007; 132(6):2131-57.

1. Inhibitors of CXCL12 and IGFBP2 for use in the treatment of pancreatic cancer associated with diabetic or prediabetic conditions.

2. Inhibitors of CXCL12 and IGFBP2 for the use according to claim 1, where prediabetes is impaired glucose tolerance or impaired fasting glucose level.

3. Inhibitors for the use of under claim 1, where the inhibitors are substances with direct inhibition of CXCL12 and IGFBP2.

4. Inhibitors for the use of claim 1, where the inhibitors are the inhibitors of the receptors of CXCL12 and IGFBP2.

5. Inhibitors for the use of claim 4, where the inhibitors are the inhibitors of CXCR4.

6. Inhibitors for the use according to claim 1, where the inhibitors are the inhibitors of signal transduction pathways of CXCL12 and IGFBP2.

7. Use of inhibitors according to claim 1 for the manufacture of pharmaceutical compositions for the treatment of pancreatic cancer associated with diabetes or prediabetes.

8. Drug preparations which contain inhibitors according to claim 1 in combination with one or more pharmaceutically acceptable carrier vehicle or auxiliary ingredient.

9. Inhibitors for the use according to claim 2, where the inhibitors are the inhibitors of signal transduction pathways of CXCL 12 and IGFBP2.

10. Inhibitors for the use according to claim 3, where the inhibitors are the inhibitors of signal transduction pathways of CXCL 12 and IGFBP2.

11. Inhibitors for the use according to claim 4, where the inhibitors are the inhibitors of signal transduction pathways of CXCL 12 and IGFBP2.

12. Inhibitors for the use according to claim 5, where the inhibitors are the inhibitors of signal transduction pathways of CXCL 12 and IGFBP2.

13. Use of inhibitors according to claim 2 for the manufacture of pharmaceutical compositions for the treatment of pancreatic cancer associated with diabetes or prediabetes.

14. Use of inhibitors according to claim 3 for the manufacture of pharmaceutical compositions for the treatment of pancreatic cancer associated with diabetes or prediabetes.

15. Use of inhibitors according to claim 4 for the manufacture of pharmaceutical compositions for the treatment of pancreatic cancer associated with diabetes or prediabetes.

16. Use of inhibitors according to claim 5 for the manufacture of pharmaceutical compositions for the treatment of pancreatic cancer associated with diabetes or prediabetes.

17. Use of inhibitors according to claim 6 for the manufacture of pharmaceutical compositions for the treatment of pancreatic cancer associated with diabetes or prediabetes.

18. Drug preparations which contain inhibitors according to claim 2 in combination with one or more pharmaceutically acceptable carrier vehicle or auxiliary ingredient.

19. Drug preparations which contain inhibitors according to claim 3 in combination with one or more pharmaceutically acceptable carrier vehicle or auxiliary ingredient.

20. Drug preparations which contain inhibitors according to claim 4 in combination with one or more pharmaceutically acceptable carrier vehicle or auxiliary ingredient.
